(19) United States
(12) Patent Application Publication
(10) Pub. No.: US 2011/0158930 A1
(43) Pub. Date: Jun. 30, 2011

(54) METHOD FOR TREATMENT OF IRRITABLE BOWEL SYNDROME

(75) Inventors: Takuya Hirata, Tokyo (JP); Toshiyuki Funatsu, Tokyo (JP); Yoshihiro Keto, Tokyo (JP); Shinobu Akuzawa, Tokyo (JP)

(73) Assignee: ASTELLAS PHARMA INC., Tokyo (JP)

(21) Appl. No.: 13/061,384

(22) PCT Filed: Aug. 27, 2009
(86) PCT No.: PCT/JP2009/064904

§ 371 Date: Feb. 28, 2011

Related U.S. Application Data
(60) Provisional application No. 61/190,559, filed on Aug. 28, 2008.

(51) Int. Cl. A61K 31/78; C07D 403/06; A61P 1/00; A61P 1/12

(52) U.S. Cl. 424/78.01; 548/302.7

(57) ABSTRACT

A method for treatment of a patient suffering from irritable bowel syndrome with diarrhea or mixed irritable bowel syndrome, which comprises administering to the patient a therapeutically effective amount of ramosetron or a pharmaceutically acceptable salt thereof in combination with a therapeutically effective amount of polycarbophil or a pharmaceutically acceptable salt thereof.

### TECHNICAL FIELD

The present invention relates to a treatment method of using ramosetron or a pharmaceutically acceptable salt thereof in combination with polycarbophil or a pharmaceutically acceptable salt thereof for treatment of a patient suffering from irritable bowel syndrome with diarrhea or mixed irritable bowel syndrome.

### BACKGROUND ART

Irritable bowel syndrome (IBS) is a functional gastrointestinal disorder with which the patient experiences chronic disturbance of bowel habit (constipation or diarrhea) and abdominal pain/discomfort without any organic disease in the gastrointestinal tract. According to the diagnostic criteria for IBS (Rome III Criteria) published in 2006, IBS is categorized as IBS with constipation, IBS with diarrhea, mixed IBS or unclassified IBS (for example, see Non-Patent Reference 1). It is reported that the prevalence of IBS to the total population reaches 10% to 20% and IBS patients account for about 20 to 40% of outpatients in gastrointestinal medicine department, and it is considered that IBS is a gastrointestinal disorder recognized at high frequency. Further, the IBS symptom greatly worsens the quality of life and the work productivity of the patient, which is being a serious social problem. At present, for the pharmacological treatment of IBS, mainly used is an antispastic drug such as anticholinergic drug, an antidiarrheal drug such as opioid receptor agonist, or a bulking-agent or a probiotics for regulating the enteric environment.

Ramosetron is a serotonin (5-HT)₃ receptor antagonist having a chemical name of (−)-(R)-5-[(1-methyl-1H-indol-3-yl)carbonyl]-4,5,6,7-tetrahydro-1H-benzimidazole (for example, see Patent Reference 1). Heretofore, ramosetron has been prescribed for an adult patient with the gastrointestinal symptom such as vomiting caused by antineoplastic agents, in a mode of oral administration at a dose of 0.1 mg once a day or intravenous injection at a dose of 0.3 mg once a day. Recently, it has been confirmed that ramosetron hydrochloride at an extremely low daily dose of 0.001 to 0.05 mg is also effective for treatment of IBS with diarrhea (for example, see Patent Reference 2). At present, there are only a few reports showing the efficacy of the combined use of 5-HT₃ receptor antagonist with any other agent for IBS, such as a noradrenaline reuptake inhibitor and a mast-cell degranulation inhibitor (for example, see Patent References 4 and 5), while it is still unclear whether the concomitant administration of ramosetron with the existing agents for IBS described above show the beneficial synergistic effect.

On the other hand, polycarbophil is a polyacrylic acid crosslinked with divinyl glycol, and is a high-molecular polymer having high water absorbability (for example, see Patent Reference 3). Polycarbophil is used as an antidiarrheal drug or for treatment of altered bowel habit in IBS. Specifically, it is considered that, owing to its water absorbing effect, polycarbophil could absorb water in an intestinal lumen increased by stress or the like, therefore normalizing the stool condition to prevent diarrhea (for example, see Non-Patent References 2 and 3).

### PRIOR ART REFERENCES

### Patent References

Patent Reference 1: EP 381422
Patent Reference 2: US 2005/0192329
Patent Reference 3: U.S. Pat. No. 3,297,664
Patent Reference 4: WO 2004/062623
Patent Reference 5: WO 2008/096777

### Non-Patent References

Non-Patent Reference 1: Gastroenterology, 2006; 130: p. 1480-1491
Non-Patent Reference 2: Japanese Journal of Pharmacology, 2002; 89: p. 133-141
Non-Patent Reference 3: Neurogastroenterology and Motility, 2008; 20(5): p. 557-565

### DISCLOSURE OF THE INVENTION

The present inventors have assiduously studied for the purpose of creating a more excellent treatment method for patients suffering from IBS with diarrhea or mixed IBS.

As a result, the inventors have confirmed an excellent synergistic effect of ramosetron in combined administration with polycarbophil both in the pharmacological efficacy and the side effect thereof, and have completed the present invention.

Using a restraint stress induced rat diarrheal model and a 5-HT induced mouse diarrheal model, the inventors investigated the synergistic effect of ramosetron and polycarbophil and have found that the combined use of the two drugs significantly enhances the respective inhibitory effects for diarrhea. Further, the inventors have found that the repeated combined administration of ramosetron and polycarbophil attenuates the inhibitory effect of ramosetron on spontaneous defecation in mice. On the other hand, it is reported that a high-molecular polymer such as colestimide or sevelamer hydrochloride adsorbs the other low-molecular compound administered simultaneously therewith and attenuates the absorption and the effect of the compound (Kays et al., Am. J. Kidney Dis. 2003; 42: 1253-1259; Yamada et al., Pharmacology and Therapy, 2001; 29: 37-44). However, oral administration of a prepared mixed solution of ramosetron and polycarbophil has been confirmed to possess the antidiarrheal effect, but on the contrary, rather provides significant effect enhancement; and therefore, it is considered that the possibility that polycarbophil may adsorb ramosetron to attenuate the effect thereof would be low.

### SUMMARY OF THE INVENTION

Specifically, the invention relates to the following:

[1] A method for treatment of a patient suffering from irritable bowel syndrome with diarrhea or mixed irritable bowel syndrome, which comprises administering to the patient a therapeutically effective amount of ramosetron or a pharmaceutically acceptable salt thereof in combination with a therapeutically effective amount of polycarbophil or a pharmaceutically acceptable salt thereof;

[2] The method of [1] comprising administering to the patient 0.001 to 0.05 mg of ramosetron hydrochloride as a daily dose or an equivalent molar amount of ramosetron or other pharmaceutically acceptable salt thereof; and

[3] The method of [1] or [2] comprising administering to the patient 1.0 to 5.0 g of polycarbophil calcium as a daily dose or an equivalent molar amount of polycarbophil or other pharmaceutically acceptable salt thereof.

The invention also relates to the following:

[4] Use of ramosetron or a pharmaceutically acceptable salt thereof for the manufacture of a medicament for the treatment of irritable bowel syndrome with diarrhea or mixed irritable bowel syndrome in combination with polycarbophil or a pharmaceutically acceptable salt thereof; and

[5] Use of ramosetron hydrochloride in a daily dose of 0.001 to 0.05 mg, or an equivalent molar amount of ramosetron or other pharmaceutically acceptable salt thereof, for the manufacture of a medicament for the treatment of irritable bowel syndrome with diarrhea or mixed irritable bowel syndrome in combination with polycarbophil or a pharmaceutically acceptable salt thereof.

The invention further relates to the following:

[6] A pharmaceutical composition comprising ramosetron or a pharmaceutically acceptable salt thereof and polycarbophil or a pharmaceutically acceptable salt thereof;

[7] The pharmaceutical composition of [6] comprising a) 0.001 to 0.05 mg of ramosetron hydrochloride as a daily dose or an equivalent molar amount of ramosetron or other pharmaceutically acceptable salt thereof, and b) 1.0 to 5.0 g of polycarbophil calcium as a daily dose or an equivalent molar amount of polycarbophil or other pharmaceutically acceptable salt thereof; and

[8] The pharmaceutical composition of [6] or [7] for use for the treatment of irritable bowel syndrome with diarrhea or mixed irritable bowel syndrome.

The invention further relates to the following:

[9] An enhancer of the effect of polycarbophil or a pharmaceutically acceptable salt thereof for improving the diarrhea symptom of irritable bowel syndrome, which comprises ramosetron or a pharmaceutically acceptable salt thereof; and

[10] The enhancer of [9], comprising 0.001 to 0.05 mg of ramosetron hydrochloride as a daily dose or an equivalent molar amount of ramosetron or other pharmaceutically acceptable salt thereof.

### ADVANTAGE OF THE INVENTION

According to the invention, there is provided a more excellent treatment method for patients suffering from IBS with diarrhea or mixed IBS.

Specifically, combined use of ramosetron or a pharmaceutically acceptable salt thereof and polycarbophil or a pharmaceutically acceptable salt thereof provides a beneficial effect on IBS symptoms even to the patients who could not obtain sufficient symptomatic improvement by the individual drugs alone. Further, the combined use with polycarbophil or a pharmaceutically acceptable salt thereof can inhibit the occurrence of constipation as the side effect in relation to the administration of ramosetron or a pharmaceutically acceptable salt thereof.

### BRIEF DESCRIPTION OF THE DRAWINGS

FIG. 1 includes graphs showing the effect of ramosetron, polycarbophil and their combination for restraint stress-induced diarrhea in rats. A shows the incidence of diarrhea in each group with n=12, in which *P<0.05/3, **P<0.01/3 to distilled water administration group (Fisher's exact test). B shows the incidence of diarrhea in each group with n=24, in which *P<0.05, **P<0.01 to distilled water administration group, #P<0.05, ##P<0.01 to ramosetron or polycarbophil administration group (Fisher's exact test).

FIG. 2 includes graphs showing the effect of ramosetron, polycarbophil and their combination for 5-HT-induced diarrhea in mice. A shows the incidence of diarrhea in each group with n=12, in which *P<0.05/3, **P<0.01/3 to distilled water administration group (Fisher's exact test). B shows the incidence of diarrhea in each group with n=24, in which *P<0.05, **P<0.01 to distilled water administration group, #P<0.05, ##P<0.01 to ramosetron or polycarbophil administration group (Fisher's exact test).

FIG. 3 shows a graph indicating the influence of single-administration of ramosetron or polycarbophil alone on spontaneous defecation in mice, in which the data indicate the mean value±SEM in each group with n=10, and *P<0.05 to distilled water administration group (Dunnett's test).

FIG. 4 shows a graph indicating the influence of single administration of ramosetron, polycarbophil or their combination on spontaneous defecation in mice, in which the data indicate the mean value±SEM in each group with n=10 to 14, and #P<0.05 to distilled water alone administration group (Student's t-test).

FIG. 5 shows a graph indicating the influence of repeated administration of ramosetron, polycarbophil or their combination on spontaneous defecation in mice, in which the data indicate the mean value±SEM in each group with n=10, #P<0.05 to distilled water alone administration group (Student's t-test), and *P<0.05 to distilled water+ramosetron administration group (Dunnett's test (multigroup)).

### BEST MODE FOR CARRYING OUT THE INVENTION

The invention is described in more detail hereinunder.

Ramosetron and its pharmaceutically acceptable salt may be readily obtained by the method described in Patent Reference 1 or according to it.

The pharmaceutically acceptable salt of ramosetron concretely includes mineral acid salts with hydrochloric acid, sulfuric acid, phosphoric acid, hydrobromic acid, etc.; salts with organic acids such as acetic acid, oxalic acid, succinic acid, citric acid, maleic acid, malic acid, fumaric acid, tartaric acid, methanesulfonic acid, etc.; salts with acidic amines such as glutamic acid, aspartic acid, etc. Above all, preferred is commercially-available ramosetron hydrochloride.

Polycarbophil and its pharmaceutically acceptable salt may be readily obtained by the method described in Patent Reference 3 or according to it.

The pharmaceutically acceptable salt of polycarbophil concretely includes alkali metal salts with sodium, potassium, etc.; alkaline earth metal salts with calcium, magnesium, etc.; aluminium salts; ammonium salts; salts with organic amines such as benzathine, choline, diethanolamine, ethylenediamine, meglumine, diethylamine, piperazine, tromethamine, procaine, etc. Above all, preferred is commercially-available polycarbophil calcium.

Ramosetron or its pharmaceutically acceptable salt and polycarbophil or its pharmaceutically acceptable salt may be, either separately or simultaneously, orally administered as a pharmaceutical composition thereof with an organic or inorganic carrier, vehicle and other additive suitable to oral administration. In case where the active ingredients are separately formulated in different preparations, those different preparations may be administered to a patient separately or simultaneously or at time intervals.

The pharmaceutical composition concretely includes tablets, powders, granules, microspheres, capsules, pills, syrups, emulsions, suspension, etc.

In the oral preparation, the active ingredient is mixed with at least one inert diluent, for example, lactose, mannitol, glucose, microcrystalline cellulose, starch, polyvinyl pyrrolidone, magnesium metasilicate aluminate. The composition may contain, according to an ordinary method, any other additive than the inert diluent, for example, a binder such as hydroxypropyl cellulose, hydroxypropylmethyl cellulose; a lubricant such as magnesium stearate, calcium stearate, polyethylene glycol, starch, talc; a disintegrant such as calcium glycolate cellulose; a stabilizer such as lactose; a dissolution promoter such as glutamic acid, aspartic acid; a plasticizer such as Tween 80, triacetin; a colorant such as titanium oxide, iron sesquioxide. Tablets or pills may be optionally coated with a sugar coating film or a film of a gastric or enteric substance such as sucrose, gelatin, agar, pectin, hydroxypropyl cellulose, hydroxypropylmethyl cellulose phthalate, etc.

The pharmaceutical composition may be an oral disintegrable tablet. For example, oral disintegrable tables may be prepared according to U.S. Pat. No. 5,466,464, U.S. Pat. No. 5,576,014, U.S. Pat. No. 6,589,554, WO2003/009831, WO2002/092057, etc.

Ramosetron is used in an extremely low dose, and is therefore especially preferably in the form of a preparation specifically stabilized to temperature and humidity. For example, as described in US2005/0026981 A1, a specific compound having a carbonyl group may be added thereto to stabilize ramosetron to temperature and humidity. The specific compound having a carbonyl group includes, concretely, aliphatic carboxylic acids (in detail, saturated or unsaturated, linear or branched aliphatic mono-, di or tri-carboxylic acid, especially aliphatic carboxylic acids having from 3 to 36 carbon atoms) or their esters; hydroxycarboxylic acids (in detail, saturated or unsaturated, linear or branched aliphatic hydroxy-mono-, di- or tri-carboxylic acids, especially hydroxycarboxylic acids having from 3 to 36 carbon atoms) or their esters; acidic amino acids, enol acids, aromatic carboxyl compounds (in detail, aromatic mono-, di or tri-carboxylic acids optionally substituted with an alkyl group having 1 to 4 carbon atoms or a hydroxy group, especially aromatic carboxylic acids having from 7 to 20 carbon atoms) or their esters, and polymer substances having a carboxyl group. One or more these compounds may be used here either singly or as combined. Not specifically defined, the amount to be used of the compound to stabilize ramosetron or its pharmaceutically acceptable salt may be any one capable of attaining the stabilization. For example, in the preparation, the amount may be from 0.01 to 90% by weight, preferably from 0.01 to 50% by weight, more preferably from 0.1 to 10% by weight in consideration of the producibility of the preparation.

Polycarbophil is highly sticky, and therefore, in case where it is orally administered as a tablet, preferably the tablet can be fully disintegrated in the stomach and can be uniformly dispersed therein. For example, as a chewable tablet, the preparation may be chewed by a patient in its mouth and the thus-disintegrated preparation may be introduced into the stomach. In addition, as described in EP 488139, a cellulose derivative may be incorporated in a preparation of polycarbophil to thereby make the preparation disintegrable under the acidic condition in a stomach. Not specifically defined, the amount to be added of the cellulose derivative may be any one capable attaining the disintegration of the preparation under the acidic condition; and for example, the amount is from 1 to 80% by weight of polycarbophil calcium in the preparation.

The dose (daily dose) of ramosetron or its pharmaceutically acceptable salt and polycarbophil or its pharmaceutically acceptable salt may be suitably determined case by case, depending on the symptom of the disease, the age, the race and the sex of the object intended for administration, etc. The daily dose of a drug as referred to herein means the total amount of the drug to be administered within 24 hours, and the total amount may be administered in a mode of single dose (one time administration a day) or multiple dose (two or more times administration within 24 hours, and the total of the multiple dose is within the range of the daily dose as referred to herein).

Ramosetron hydrochloride may be administered to a patient with IBS generally in a daily dose of about from 0.001 to 0.05 mg/adult in a mode of ordinary oral administration, most preferably in a daily dose of from 0.0025 to 0.01 mg; and this is orally administered once a day. Polycarbophil calcium may be administered generally in a daily dose of about from 1 to 8 g/adult in a mode of ordinary oral administration, most preferably in a daily dose of from 1.5 to 3.0 g; and this is orally administered along with water, once or as divided in two to four times a day.

Accordingly, for example, polycarbophil calcium may be administered three times a day after every meal each in a dose of from 0.5 to 1.0 g, and in one time of those, ramosetron hydrochloride may be administered as combined in a dose of from 0.0025 to 0.01 mg.

Various treatment plans may be employed. Some patients may be treated occasionally. For example, ramosetron or its pharmaceutically acceptable salt and polycarbophil or its pharmaceutically acceptable salt are administered each in the above-mentioned daily dose to the patient who has developed a sudden onset of IBS symptoms, during a sufficient period of time for relieving the symptom of IBS. For example, ramosetron or its pharmaceutically acceptable salt and polycarbophil or its pharmaceutically acceptable salt are administered to the patient each in the above-mentioned daily dose for a period of 7 days, 14 days, 21 days, 28 days, 6 weeks, 8 weeks, 12 weeks, 16 weeks or longer until the symptom of IBS could disappear. Some other patients may be treated continuously for a longer period of time. For example, the above-mentioned daily dose of ramosetron or its pharmaceutically acceptable salt is preventively administered occasionally or continuously during a long period of time (at least for 6, 9, 12 or 15 months) for the purpose of evading or inhibiting the onset of IBS. Further, any other treatment plans may also be employable.

According to Rome III diagnostic criteria, IBS is categorized as IBS with constipation, IBS with diarrhea, mixed IBS or unclassified IBS (Longstreth et al., Gastroenterology, 2006; 130: 1480-1491); combined administration of ramosetron or its pharmaceutically acceptable salt and polycarbophil or its pharmaceutically acceptable salt is considered effective for the diarrheal symptom of patients suffering from IBS with diarrhea and mixed IBS.

### EXAMPLES

The invention is described in more detail with reference to the following Examples and Experimental Examples; however, the invention is not limited to these Examples, etc.

### Example 1

| | | |
|---|---|---|
| Ramosetron hydrochloride | 0.0008 | parts |
| Mannitol | 89 | parts |
| Citric anhydride | 0.1 | parts |
| Maltose | 10 | parts |
| Red iron sesquioxide | 1 | part |
| Magnesium stearate | 1 | part |

Ten parts of maltose, 0.0008 parts of ramosetron hydrochloride, 0.1 parts of citric anhydride and 1 part of red iron sesquioxide were suspended in 67 parts of water with stirring with a magnetic stirrer to prepare a spray liquid (concentration, 15% by weight). Next, 89 parts of mannitol was fed into a fluidized bed granulator (FLOW COATER, by Freund), and the above spray liquid was sprayed onto it at a spraying speed of 10 g/min for fluidized bed granulation. After the granulation, the granulated product was dried at a suction temperature of 40° C. for 5 minutes, and then mixed with 1 part of magnesium stearate. Using a rotary tabletter, the mixed powder was tabletted into tablets each weight 120 mg and having an initial hardness of about 1 kp. This was stored at 25° C. and at a relative humidity of 75% for 18 hours, and then stored at 30° C. and at a relative humidity of 40% for 4 hours, thereby giving oral disintegrable tablets.

### Example 2

| | | |
|---|---|---|
| Ramosetron hydrochloride | 0.0008 | parts |
| Mannitol | 88 | parts |
| Maltose | 10 | parts |
| Yellow iron sesquioxide | 1 | part |
| Citric anhydride | 0.2 | parts |
| Magnesium stearate | 1 | part |

Ten parts of maltose, 0.0008 parts of ramosetron hydrochloride, 1 part of red iron sesquioxide and 0.2 parts of citric anhydride were suspended in 67 parts of water with stirring with a magnetic stirrer to prepare a spray liquid (concentration, 15% by weight). Next, 88 parts of mannitol was fed into a fluidized bed granulator (FLOW COATER, by Freund), and the above spray liquid was sprayed onto it at a suction temperature of 50° C. and at a spraying speed of 10 g/min in a cycle of spraying/drying/shaking of 15 seconds/15 seconds/10 seconds for fluidized bed granulation. After the granulation, the granulated product was dried at a suction temperature of 40° C. for 5 minutes, and then mixed with 1 part of magnesium stearate. Using a rotary tabletter, the mixed powder was tabletted into tablets each weight 120 mg and having an initial hardness of about 1 kp. This was stored at 25° C. and at a relative humidity of 75% for 18 hours, and then stored at 30° C. and at a relative humidity of 40% for 4 hours, thereby giving oral disintegrable tablets.

### Example 3

| Polycarbophil calcium | 62.5 | parts |
|---|---|---|
| Carboxymethyl cellulose | 1.25 | parts |
| Crystalline cellulose | quantum libet | |
| Magnesium stearate | 0.6 | parts |
| Hydroxypropylmethyl cellulose | 2 | parts |
| Macrogol 6000 | 0.5 | parts |
| Titanium oxide | 0.5 | parts |

A part (about ½ of the total amount) of carboxymethyl cellulose was added to polycarbophil calcium and mixed at room temperature, then granulated with water in an amount of 5% by weight of polycarbophil, and dried at 60° C. for about 10 hours. The granulated product was regulated for particle size through a 18-mesh sieve, and then the remaining carboxymethyl cellulose and crystalline cellulose were added thereto, and further magnesium stearate was added thereto to give a powder to be tabletted. This was tabletted into tablets each containing 625 mg of polycarbophil calcium in one tablet. The tables were coated with a film of hydroxypropylmethyl cellulose, Macrogol 6000 and titanium oxide to be film-coated tablets.

### Experimental Examples

### I. Test Method:

### (1) Material and Method:

The animals were housed under free drinking and free feeding condition in a temperature (22±2° C.) and humidity (55±5%)-controlled animal room in a dark-light cycle of 12 hours. All the animal experiments were carried out under the approval by Animal Ethical Committee of Astellas Pharma.

Ramosetron hydrochloride, polycarbophil calcium (Astellas Pharma, Inc.) and 5-HT creatinine sulfate (Wako Pure Chemical Industries Ltd.) were used. Ramosetron was dissolved and diluted with distilled water. Polycarbophil was prepared by decalcification of polycarbophil calcium according to a reported method (Yamada et al., Iyakuhin Kenkyu 1997; 28: 23-32), and suspended and diluted with distilled water. In this experiment, ramosetron was used and expressed as its hydrochloride form. All the test substances were orally administered in a dose of 12 mL/kg. In combined administration of ramosetron and polycarbophil, a mixed solution of the two drugs was prepared and this was orally administered.

### (2) Effect for Restraint Stress Induced Diarrhea in Rats:

Male Wistar rats (13 to 14-week age, Japan SLC Inc.) that had been fasted overnight were stressed by confinement in a restraint stress cage (KN-468, Natsume Seisakusho Co., Ltd.) (Hirata et al., Neurogastroenterol. Motil. 2008; 20: 557-565). The condition of the stool excreted within 3 hours after the initiation of the restraint stress was observed, and the occurrence of diarrhea was defined in case where the excreted stool condition was wet, amorphous and muddy stool. The effect of ramosetron or polycarbophil alone was investigated with the following group composition (n=12 in every group): Distilled water administration group (control group); ramosetron administration group (0.1, 0.3 and 1 μg/kg); polycarbophil administration group (100, 300 and 1,000 mg/kg). The effect of combined administration of ramosetron and polycarbophil was investigated with the following group composition (n=24 in every group): Distilled water administration group (control group); ramosetron administration group (0.3 μg/kg); polycarbophil administration group (300 mg/kg); ramosetron (0.3 μg/kg)+polycarbophil (300 mg/kg) administration group. All the test substances were orally administered to the rat 4 hours before the initiation of the restrained stress.

### (3) Effect for 5-HT Induced Mouse Diarrhea:

Each test substance was orally administered to male ICR mice (9-week age, Clea Japan, Inc.), and after 2 hours, 5-HT (3 mg/kg) was intraperitoneally administered (Miyata et al., J. Pharmacol. Exp. Ther. 1992; 261: 297-303). The mice were put individually in separate cages, and the condition of the stool excreted within 3 hours after the 5-HT administration was observed. The occurrence of diarrhea was defined in case where the excreted stool condition was loose stool (wet pasty stool) or watery stool (amorphous muddy stool). The effect of ramosetron or polycarbophil alone was investigated with the following group composition (n=12 in every group): Distilled water administration group (control group); ramosetron administration group (10, 30 and 100 μg/kg); polycarbophil administration group (100, 300 and 1,000 mg/kg). The effect of combined administration of ramosetron and polycarbophil was investigated with the following group composition (n=24 in every group): Distilled water administration group (control group); ramosetron administration group (30 μg/kg); polycarbophil administration group (300 mg/kg); ramosetron (30 μg/kg)+polycarbophil (300 mg/kg) administration group.

### (4) Effect on Spontaneous Defecation in Mice:

In this experiment, male ddy mice (Nippon SLC, 5-week age) were used. Spontaneous defecation was measured individually in separate cages with a wire flooring under free drinking and free feeding condition. To avoid the novelty stress, the mice were individually housed in the respective cages with a wire flooring, from one day before the test date.

The effect of single administration of ramosetron or polycarbophil alone was investigated with the following group composition, and the total stool weight in 2 hours after administration of each test substance was measured (n=10 in every group): Distilled water administration group (control group); ramosetron administration group (10, 30 and 100 μg/kg); polycarbophil administration group (100, 300 and 1,000 mg/kg).

The effect of combined single administration of ramosetron and polycarbophil was investigated with the following group composition, and the total stool weight in 2 hours after administration of each test substance was measured (n=10 to 14 in every group): Distilled water administration group (control group); polycarbophil administration group (300 and 1,000 mg/kg); distilled water+ramosetron (100 μg/kg) administration group; polycarbophil (300 and 1,000 mg/kg)+ramosetron (100 μg/kg) administration group.

The effect of combined repeated administration of ramosetron and polycarbophil was investigated by orally administering to the mice the following test substance three times a day (morning, noon, evening) for 4 days followed by measuring the total weight of the stool excreted for 2 hours after the drug administration at the noon on day 4 (n=10 in every group): Distilled water administration group (control group, t.i.d.); polycarbophil administration group (300 and 1,000 mg/kg, t.i.d.), distilled water (t.i.d.)+ramosetron [100 μg/kg, q.d. (at noon)] administration group; polycarbophil (300 and 1,000 mg/kg, t.i.d.)+ramosetron [100 μg/kg, q.d. (at noon)] administration group.

In every administration protocol, spontaneous defecation was measured from 1 h after the animal room changed light period. Specifically, in the experiment of repeated administration, the lighting cycle in the animal room was so modified that the light period start from about 1 hour before the day-time administration, and under the condition, the animals were habituated (1 week), and then used for administration and measurement.

### (5) Statistical Analysis:

All the results were statistically analyzed using Statistical Analysis System ver. 8.2 (SAS Institute Japan). In this experiment, all the data were expressed as two significant figures. In the investigation using the diarrheal models, the incidence of diarrhea was calculated in each group, then the significance was evaluated according to the Fisher's exact test, and if desired, the significance level was corrected according to the Bonferroni's method for consideration of multiplicity (the case of P<0.05 or P<0.05/3 was given significance). In the experiments of spontaneous defecation, the mean value of total stool weight±SEM was calculated in every group, and the significance was evaluated according to Student's t-test (between two groups) or Dunnett's multiple comparison test (between multiple groups) (the case of P<0.05 was given significance).

### II. Results:

### 1. Effect for Restraint Stress Induced Diarrhea in Rats:

In the control group, 3-hour restraint stress caused diarrhea at the incidence of higher than 90% in rats. Oral administration of ramosetron (0.1 to 1 μg/kg) and polycarbophil (100 to 1,000 mg/kg) alone dose-dependently inhibited restraint stress induced diarrhea, and provided a significant inhibitory effect with dosage of 0.3 and 1 μg/kg, and 300 and 1,000 mg/kg, respectively (FIG. 1A).

The incidence of diarrhea in 0.3 μg/kg of ramosetron or 300 mg/kg of polycarbophil alone was 46% and 58%, respectively (FIG. 1B). On the other hand, the incidence of diarrhea in combined administration of the two was 13%; and a significant difference was observed between the combined administration group and the respective mono-administration group (FIG. 1B).

### 2. Effect for 5-HT Induced Diarrhea in Mice:

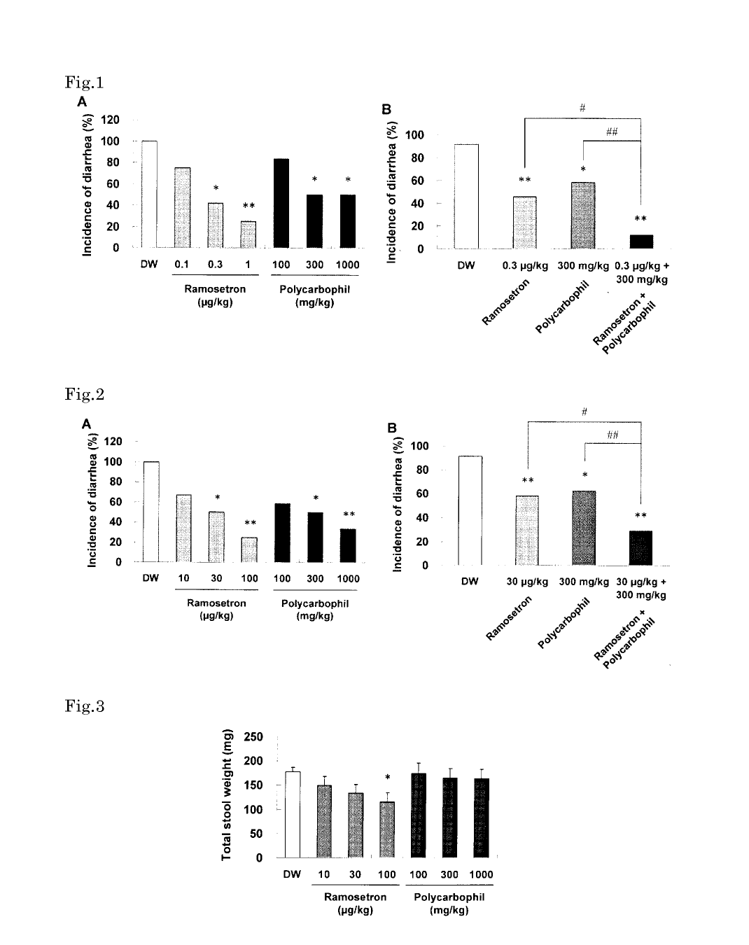

In the control group, intraperitoneal administration of 5-HT (3 mg/kg) caused diarrhea at the incidence of higher than 90% in mice. Oral administration of ramosetron (10 to 100 μg/kg) and polycarbophil (100 to 1,000 mg/kg) alone dose-dependently inhibited 5-HT induced diarrhea, and provided a significant inhibitory effect with dosage of 30 and 100 μg/kg, and 300 and 1,000 mg/kg, respectively (FIG. 2A).

The incidence of diarrhea in 30 μg/kg of ramosetron or 300 mg/kg of polycarbophil alone was 58% and 63%, respectively (FIG. 2B). On the other hand, the incidence of diarrhea in combined administration of the two was 29%; and a significant difference was observed between the combined administration group and the respective mono-administration group (FIG. 2B).

### 3. Effect for Spontaneous Defecation in Mice:

In the control group, the stool weight of spontaneous defecation in the mice for 2 hours after the oral administration of distilled water was ranged from 140±22 to 190±23 mg (FIGS. 3, 4 and 5). Oral administration of ramosetron (10 to 100 μg/kg) alone dose-dependently inhibited spontaneous defecation; and in the 100 μg/kg administration group, a significant reduction in the total stool weight was observed as compared with that in the distilled water administration group (FIG. 3). On the other hand, oral administration of polycarbophil (100 to 1,000 mg/kg) alone had no significant influence on spontaneous defecation (FIG. 3).

The total stool weight for 2 hours after single combined administration of 300 or 1,000 mg/kg of polycarbophil and 100 μg/kg of ramosetron was on the same level as that in single administration of 100 μg/kg of ramosetron alone; and there was not recognized any significant difference between the two groups (FIG. 4).

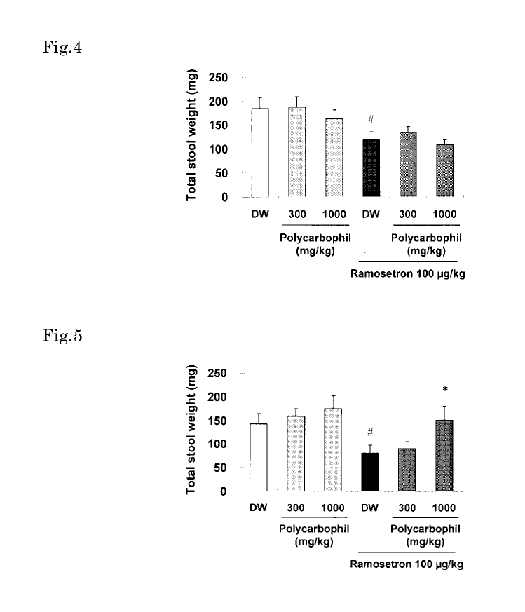

Four-day repeated oral administration of polycarbophil, 300 and 1,000 mg/kg (t.i.d.) alone slightly increased the stool weight in spontaneous defecation in mice; however, at any dose, no significant difference was observed between the polycarbophil group and the distilled water group (FIG. 5). On the other hand, repeated combined administration of polycarbophil, 300 or 1,000 mg/kg (t.i.d.), and ramosetron, 100 μg/kg (q.d.) attenuated the inhibitory effect of ramosetron on spontaneous defecation; and in the repeated combined administration group with ramosetron and polycarbophil, 1,000 mg/kg (t.i.d.), a significant increase was observed as compared with that in the repeated administration group with ramosetron alone (FIG. 5).

### INDUSTRIAL APPLICABILITY

According to the invention, there is provided a more excellent treatment method for patients suffering from IBS with diarrhea or mixed IBS.

1. A method for treatment of a patient suffering from irritable bowel syndrome with diarrhea or mixed irritable bowel syndrome, which comprises administering to the patient a therapeutically effective amount of ramosetron or a pharmaceutically acceptable salt thereof in combination with a therapeutically effective amount of polycarbophil or a pharmaceutically acceptable salt thereof.

2. The method of claim 1 comprising administering to the patient 0.001 to 0.05 mg of ramosetron hydrochloride as a daily dose or an equivalent molar amount of ramosetron or other pharmaceutically acceptable salt thereof.

3. The method of claim 1 comprising administering to the patient 1.0 to 5.0 g of polycarbophil calcium as a daily dose or an equivalent molar amount of polycarbophil or other pharmaceutically acceptable salt thereof.

4. Use of ramosetron or a pharmaceutically acceptable salt for the manufacture of a medicament for the treatment of irritable bowel syndrome with diarrhea or mixed irritable bowel syndrome in combination with polycarbophil or a pharmaceutically acceptable salt thereof.

5. Use of ramosetron hydrochloride in a daily dose of 0.001 to 0.05 mg, or an equivalent molar amount of ramosetron or other pharmaceutically acceptable salt thereof, for the manufacture of a medicament for the treatment of irritable bowel syndrome with diarrhea or mixed irritable bowel syndrome in combination with polycarbophil or a pharmaceutically acceptable salt thereof.

6. A pharmaceutical composition comprising ramosetron or a pharmaceutically acceptable salt thereof and polycarbophil or a pharmaceutically acceptable salt thereof.

7. The pharmaceutical composition of claim 6, comprising a) 0.001 to 0.05 mg of ramosetron hydrochloride as a daily dose or an equivalent molar amount of ramosetron or other pharmaceutically acceptable salt thereof, and b) 1.0 to 5.0 g of polycarbophil calcium as a daily dose or an equivalent molar amount of polycarbophil or other pharmaceutically acceptable salt thereof.

8. The pharmaceutical composition of claim 7, wherein the composition is used for the treatment of irritable bowel syndrome with diarrhea or mixed irritable bowel syndrome.

9. An enhancer of effect of polycarbophil or a pharmaceutically acceptable salt thereof for improving diarrhea symptom of irritable bowel syndrome, which comprises ramosetron or a pharmaceutically acceptable salt thereof.

10. The enhancer of claim 9, comprising 0.001 to 0.05 mg of ramosetron hydrochloride as a daily dose or an equivalent molar amount of ramosetron or other pharmaceutically acceptable salt thereof.
